(19) United States
(12) Patent Application Publication
(10) Pub. No.: US 2025/0292201 A1
(43) Pub. Date: Sep. 18, 2025

(54) APPARATUS AND METHOD FOR MEDICINE INFORMATION MANAGEMENT, AND MEDICINE MONITORING SYSTEM INCLUDING THE APPARATUS

(71) Applicant: CJ LOGISTICS CORPORATION, Seoul (KR)

(72) Inventors: Hyeon Seung YOO, Seoul (KR); Yong lk WON, Seoul (KR); Jae Hyuck CHOI, Seoul (KR)

(21) Appl. No.: 18/859,752

(22) PCT Filed: Apr. 24, 2023
(86) PCT No.: PCT/KR2023/005556

§ 371 Date: Oct. 24, 2024

(30) Foreign Application Priority Data
Apr. 25, 2022 (KR) 10-2022-0051075

(51) Int. Cl. G06Q 10/083; G16H 70/40

(52) U.S. Cl. CPC G06Q 10/0838; G16H 70/40

(57) ABSTRACT

Disclosed are an apparatus and method for managing medicine information, and a medicine monitoring system including the apparatus.

### TECHNICAL FIELD

The present invention relates to an apparatus and method for managing medicine information, and a medicine monitoring system including the apparatus, and more specifically, to a technique for comprehensively managing medicine information by acquiring data indicating distribution status of medicines and processing inquiry and update requests of each participant throughout the entire distribution process until the medicines are delivered to demanders through distributors and logistics companies after the medicines are produced by manufacturers.

### BACKGROUND ART

Since medicines have a characteristic of greatly affecting public health, systematic and detailed management is very important to maintain the quality of medicines in the entire distribution process from production (manufacturing) to final consumption, and higher-level medicine management standards are also introduced internationally.

With the recent pandemic of infectious diseases such as COVID-19, the need for a method of tracking and checking distribution status of each medicine in real-time is increasing day by day globally, as well as domestically.

However, at present, it remains at the level of fragmentarily verifying only the information one-sidedly provided by logistics companies while delivering goods such as medicines, and anyone who knows an invoice number may easily grasp the delivery status of a corresponding item. Therefore, it is vulnerable to security since the delivery status of medicines is easily exposed to an unspecified number of people, it is difficult for the suppliers of the medicines, as well as demanders who order the medicines, to confirm detailed status items for intuitively understanding whether distribution of specific medicines is appropriately proceeded. Furthermore, although a fatal situation occurs in maintaining the quality of medicines, such as breaking the cold chain condition due to a mistake of a logistics company or a systematic error in the distribution process, it is practically unable to identify a cause and take an immediate action.

### DISCLOSURE OF INVENTION

### Technical Problem

Therefore, the present invention has been made in view of the above problems, and it is an object of the present invention to provide an apparatus and method for transparently and safely managing medicine information throughout the entire distribution process until the medicines are delivered to demanders through distributors and logistics companies after the medicines are produced by manufacturers, by processing requests for inquiring and updating the medicine information received from each participant through communication with a private network that is constructed to be accessed only by participants (manufacturers, distributors, logistics companies, and demanders) who are approved to participate in the distribution process of medicines.

In addition, another object of the present invention is to provide an apparatus and method for automatically taking immediate actions (e.g., reorder medicines, guide related participants) for various problem situations that may occur during distribution of medicines by checking validity of a transaction indicating distribution status of medicines while tracking and monitoring the distribution process for each medicine.

Other objects and advantages of the present invention may be understood by the following description and will be more clearly known by the embodiments of the present invention. Furthermore, it will be easily understood that the objects and advantages of the present invention may be realized by the means indicated in the claims and combinations thereof.

### Solution to Problem

To accomplish the above objects, according to one aspect of the present invention, there is provided a medicine information management apparatus comprising: a communication unit configured to communicate with a plurality of supplier peers mapped to a plurality of suppliers, a plurality of logistics company peers mapped to a plurality of logistics companies, and a plurality of demander peers mapped to a plurality of demander peers, which are approved to participate in a private network; a control unit for performing channel allocation and authority grant for the plurality of supplier peers, the plurality of logistics company peers, and the plurality of demander peers, using a result of authentication performed on the plurality of suppliers, the plurality of logistics companies, and the plurality of demanders on the basis of participant identification data provided by the plurality of supplier peers, the plurality of logistics company peers, and the plurality of demander peers; and a storage unit for storing channel information that defines a channel to which each of the plurality of supplier peers, the plurality of logistics company peers, and the plurality of demander peers belongs as a result of performing the channel allocation, and authority information that defines an inquiry right and an update right of each of the plurality of supplier peers, the plurality of logistics company peers, and the plurality of demander peers as a result of performing the authority grant.

The control unit may be configured to generate a data stack including medicine identification data in response to an update request for registering the medicine identification data assigned to each medicine unit corresponding to a medicine order for each supplier received from each logistics company peer by a logistics company mapped to a corresponding logistics company peer. The control unit may be configured to control the communication unit to transmit the data stack to a channel to which the corresponding logistics company peer belongs so that the data stack is added to medicine transaction books of all participants of the channel to which the corresponding logistics company peer belongs.

The medicine identification data may include a serial shipping container code (SSCC) assigned to the medicine unit; and distribution status of the medicine unit. The medicine identification data may further include at least one among a name, quantity, production area, production date, export date, shipping date, expiry date, manufacturing certificate, Good Manufacturing Practice (GMP) evaluation report, and invoice of each medicine included in the medicine unit.

The control unit may be configured to verify, when a query request for the medicine transaction book is received from a requesting peer that is any one among the plurality of supplier peers, the plurality of logistics company peers, and the plurality of demander peers, an inquiry right of the requesting peer for the medicine transaction book using the authority information and the channel information. The control unit may be configured to transmit inquiry data acquired from the medicine transaction book to the requesting peer in response to the inquiry request when the inquiry right of the requesting peer is verified.

The control unit may be configured to generate medicine tracking information associated with each of the plurality of suppliers by date. The medicine tracking information may include at least one among the number of new orders, the number of update requests received for all medicine orders, the number of orders before delivery, the number of orders being delivered, and the number of orders that have been delivered.

The control unit may be configured to generate medicine tracking information for each medicine unit associated with each of the plurality of suppliers. The medicine tracking information may indicate an update event generated for the medicine unit and a time of generating the update event over a tracking period from a time of ordering the medicine unit to a present time.

The control unit may be configured to verify, in response to a request for updating distribution status of the medicine unit received from a requesting peer that is any one among the plurality of supplier peers, the plurality of logistics company peers, and the plurality of demander peers, an update right of the requesting peer using the authority information and the channel information. The control unit may be configured to update the distribution status of the medicine unit according to the update request when the update right of the requesting peer is verified. The control unit may be configured to control the communication unit to transmit a new data stack including an update result of the distribution status to a channel to which the requesting peer belongs so that the new data stack is added to the medicine transaction book. The distribution status may indicate at least one among inventory storage information, delivery vehicle dispatch information, delivery status information, and temperature information of the medicine unit.

A medicine monitoring system according to another aspect of the present invention includes the medicine information management apparatus and the private network.

According to still another aspect of the present invention, there is provided a medicine information management method comprising the step of: performing channel allocation and authority grant for a plurality of supplier peers, a plurality of logistics company peers, and a plurality of demander peers, using a result of authentication performed on a plurality of suppliers, a plurality of logistics companies, and a plurality of demanders on the basis of participant identification data provided by the plurality of supplier peers mapped to the plurality of suppliers, the plurality of logistics company peers mapped to the plurality of logistics companies, and the plurality of demander peers mapped to the plurality of demander peers, which are approved to participate in a private network; and storing channel information that defines a channel to which each of the plurality of supplier peers, the plurality of logistics company peers, and the plurality of demander peers belongs as a result of performing the channel allocation, and authority information that defines an inquiry right and an update right of each of the plurality of supplier peers, the plurality of logistics company peers, and the plurality of demander peers as a result of performing the authority grant.

The medicine information management method may further comprise the steps of: generating a data stack including medicine identification data in response to an update request for registering the medicine identification data assigned to each medicine unit corresponding to a medicine order for each supplier received from each logistics company peer by a logistics company mapped to a corresponding logistics company peer; and transmitting the data stack to a channel to which the corresponding logistics company peer belongs so that the data stack is added to medicine transaction books of all participants of the channel to which the corresponding logistics company peer belongs.

The medicine identification data may include: a serial shipping container code (SSCC) assigned to the medicine unit; and distribution status of the medicine unit. The medicine identification data may further include at least one among a name, quantity, production area, production date, export date, shipping date, expiry date, manufacturing certificate, Good Manufacturing Practice (GMP) evaluation report, and invoice of each medicine included in the medicine unit.

The medicine information management method may further comprise the steps of: receiving a query request for the medicine transaction book from a requesting peer that is any one among the plurality of supplier peers, the plurality of logistics company peers, and the plurality of demander peers; verifying an inquiry right of the requesting peer for the medicine transaction book using the authority information and the channel information; and transmitting inquiry data acquired from the medicine transaction book to the requesting peer in response to the inquiry request when the inquiry right of the requesting peer is verified.

The medicine information management method may further comprise the step of the step of generating medicine tracking information associated with each of the plurality of suppliers by date. The medicine tracking information may include at least one among the number of new orders, the number of update requests received for all medicine orders, the number of orders before delivery, the number of orders being delivered, and the number of orders that have been delivered.

The medicine information management method may further comprise the step of generating medicine tracking information for each medicine unit associated with each of the plurality of suppliers. The medicine tracking information may indicate an update event generated for the medicine unit and a time of generating the update event over a tracking period from a time of ordering the medicine unit to a present time.

The medicine information management method may further comprise the steps of: verifying, in response to a request for updating distribution status of the medicine unit received from a requesting peer that is any one among the plurality of supplier peers, the plurality of logistics company peers, and the plurality of demander peers, an update right of the requesting peer using the authority information and the channel information; updating the distribution status of the medicine unit according to the update request when the update right of the requesting peer is verified; and transmitting a new data stack including an update result of the distribution status to a channel to which the requesting peer belongs so that the new data stack is added to the medicine transaction book.

A computer program according to another aspect of the present invention executes the medicine information management method.

### Advantageous Effects of Invention

According to at least one of the embodiments of the present invention, it is possible to transparently and safely manage medicine information while maintaining quality of medicines throughout the entire distribution process until the medicines are delivered to demanders through distributors and logistics companies after the medicines are produced by manufacturers, by processing requests for inquiring and updating the medicine information received from each participant through communication with a private network that is constructed to be accessed only by participants (manufacturers, distributors, logistics companies, and demanders) who are approved to participate in the distribution process of medicines.

In addition, according to at least one of the embodiments of the present invention, as a different right is granted to each participant (manufacturer, distributor, logistics company, and demander) so that only mutually agreed participants may inquiry and update distribution information of a specific medicine, confidentiality of medicine information can be guaranteed.

In addition, according to at least one of the embodiments of the present invention, as immediate actions (e.g., cancellation of delivery and reorder of medicines, guidance to related participants) are automatically performed for various problem situations (e.g., dissatisfaction with cold chain conditions) that may occur during distribution of medicines by checking validity of a transaction indicating distribution status of medicines while tracking and monitoring the distribution status for each distribution unit of medicines, a situation of delivering medicines with a distribution accident to demanders and leading to misuse and abuse of medicines can be prevented, and new medicines with secured safety can be delivered again to the demanders.

In addition, according to at least one of the embodiments of the present invention, as delivery addresses of the same demander, which are differently managed for each supplier of medicines in the medicine transaction book shared for each channel in a private network, are unified, data indicating distribution status of medicines sent from several suppliers can be provided in an integrated manner for the same demander.

The effects of the present invention are not limited to the effects mentioned above, and unmentioned other effects will be clearly understood by those skilled in the art from the description of the claims.

### BRIEF DESCRIPTION OF DRAWINGS

Since the following drawings attached this specification illustrate, together with the detailed description of the present invention described below, preferred embodiments of the present invention, and serve further understand the technical spirit of the present invention, the present invention should not be construed as being limited only to the matters described in the drawings.

FIG. 1 is an exemplarily view showing the configuration of a medicine monitoring system according to an embodiment of the present invention.

FIG. 2 is an exemplarily view showing a channel relationship between peers in the private network shown in FIG. 1.

FIG. 3 a view showing a list of a plurality of Rest APIs and their functions provided to the application of a participant.

FIG. 4 is a view showing the structure of medicine identification data assigned for each medicine unit.

FIG. 5 is a view referenced to describe the operation of a medicine information management apparatus for managing medicine information in association with a supplier, a logistics company, and a demander belonging to the same channel in a private network.

FIG. 6 is a flowchart referenced to describe the channel allocation operation and the authority grant operation performed by the medicine information management apparatus for a peer mapped to each participant of a private network.

FIG. 7 is a flowchart referenced to describe a method of a medicine information management apparatus for processing an inquiry request received from each peer of a private network.

FIG. 8 is a flowchart referenced to describe a method of a medicine information management apparatus for processing an update request received from each peer of a private network.

FIG. 9 is a flowchart referenced to describe a method of a medicine information management apparatus for unifying delivery address information of the same demander managed by a plurality of suppliers.

FIGS. 10 to 12 are views showing medicine information screens that can be provided in response to an inquiry request received from a participant of a private network.

### MODE FOR THE INVENTION

Details of the objects and technical configurations of the present invention and operational effects according thereto will be more clearly understood by the following detailed description based on the drawings attached in the specification of the present invention. An embodiment according to the present invention will be described in detail with reference to the accompanying drawings.

The embodiments disclosed in this specification should not be construed or used as limiting the scope of the present invention. For those skilled in the art, it is natural that the description including the embodiments of the present specification have various applications. Accordingly, any embodiments described in the detailed description of the present invention are illustrative for better describing of the present invention, and are not intended to limit the scope of the present invention to the embodiments.

The functional blocks shown in the drawings and described below are merely examples of possible implementations. Other functional blocks may be used in other implementations without departing from the spirit and scope of the detailed description. In addition, although one or more functional blocks of the present invention are expressed as separate blocks, one or more of the functional blocks of the present invention may be combinations of various hardware and software configurations that perform the same function.

The terms including ordinal numbers, such as “first”, “second”, and the like, are used to distinguish any one of the various components from the others, and are not used to limit the components by such terms.

In addition, the expressions including certain components are expressions of “open type” and only refer to existence of corresponding components, and should not be construed as excluding additional components.

Furthermore, when a certain component is referred to as being “connected” or “coupled” to another component, it may be directly connected or coupled to another component, but it should be understood that other components may exist in between.

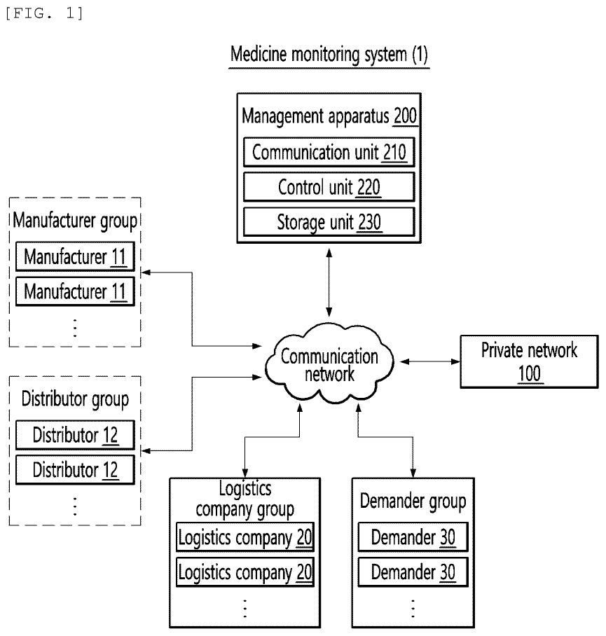

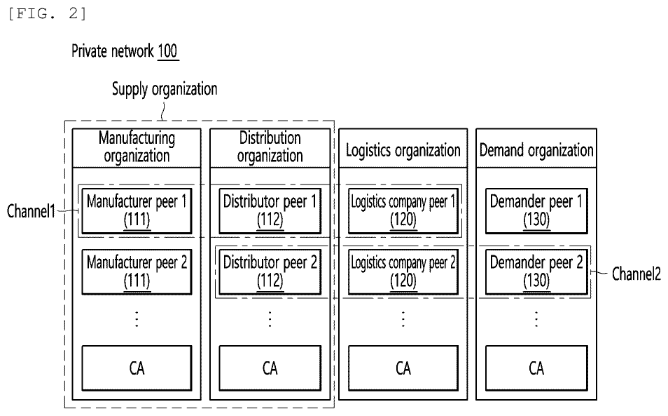

FIG. 1 is an exemplarily view showing the configuration of a medicine monitoring system 1 according to an embodiment of the present invention, and FIG. 2 is an exemplarily view showing a channel relationship between peers in the private network 100 shown in FIG. 1.

Referring to FIGS. 1 and 2, a medicine monitoring system 1 is a system for integrally recording and managing data obtained by tracking the entire distribution process from the time of manufacturing medicines to the time of completing delivery to demanders, and includes a private network 100 and a medicine information management apparatus 200.

The private network 100 includes a supply organization, a logistics organization, and a demand organization. The supply organization includes at least one among a manufacturing organization and a distribution organization.

The manufacturing organization includes a plurality of peers (hereinafter, referred to as ‘manufacturer peer 111’) matched with a plurality of manufacturers 11 of a manufacturer group. The manufacturing organization may further include a certificate authority peer (CA) for issuing and managing certificates for the plurality of manufacturers 11. Each manufacturer 11 is a subject that produces medicines tracked by the medicine monitoring system 1 and transfers the produced medicines to the distributor 12 or the logistics company 20 in medicine units packaged as a unit (e.g., box package) in response to an order request previously received from the demander 30. When the medicine unit is a box package form, a box label for identifying the medicine unit may be attached on the outer surface of the box.

The distribution organization includes a plurality of peers (hereinafter, referred to as ‘distributor peer 112’) matched with a plurality of distributors 12 of a distributor group. Each of the plurality of distributors 12 provides medicines, of which the ownership has been transferred from at least one of the plurality of manufacturers 11, to at least one of a plurality of logistics companies 20. The distribution organization may further include a certificate authority peer (CA) for issuing and managing certificates for the plurality of distributors 12.

The manufacturer 11 and the distributor 12 may be generally referred to as a supplier in that they are providers of medicines managed according to the present invention.

The logistics organization includes a plurality of peers (hereinafter, referred to as ‘logistics company peer 120’) matched with a plurality of logistics companies 20 of a logistics company group. The logistics organization may further include a certificate authority peer (CA) for issuing and managing certificates for the plurality of logistics companies 20.

The demand organization includes a plurality of peers (hereinafter, referred to as ‘demander peer’) matched with a plurality of demanders 30 of a demander group. Each of the plurality of demanders 30 is a final receiver (e.g., hospital, clinic, public health center, pharmacy) of a medicine delivered through the logistics organization after being manufactured by at least one among the manufacturing organization and the distribution organization, or a terminal used by the receiver to interwork with the demander peer 130 mapped thereto. The demand organization may further include a certificate authority peer (CA) for issuing and managing certificates for the plurality of demanders 30.

The certificate authority peer (CA) of each organization may use participant identification data provided by the participants of the same organization (e.g., business registration certificate, unique number certificate, representative certification documents) to confirm whether each participant is approved in advance to participate in the private network 100 through an authentication procedure such as SNS authentication, Decentralized Identifiers (DID), or the like.

All the components (manufacturer 11, distributor 12, logistics company 20, and demander peer 130) of the manufacturing organization, distribution organization, logistics organization, and demand organization are approved (permitted) in advance to participate in the private network 100 through peers mapped thereto. The private network 100 may be implemented based on the Hyperledger Fabric blockchain network of Amazon Web Services (AWS) in that it is a closed network in which only the subjects permitted in advance may participate.

All the peers belonging to the private network 100 described above belong to at least one channel, and two or more peers belong to each channel. Referring to FIG. 2, manufacturer peer 1, distributor peer 1, and logistics company peer 1 belong to channel 1 and share the same medicine transaction book with each other, and distributor peer 2, logistics company peer 2, and demander peer 2 belong to channel 2 and share the same medicine transaction book with each other. The channels are in an independent relationship in which a separate medicine transaction book is managed for each channel, and each peer is fundamentally blocked not to access (e.g., inquire, record, delete) the medicine transaction book managed in a channel to which the peer does not belong. When the private network 100 is implemented based on the Hyperledger Fabric blockchain network, the medicine transaction book may be referred to as a ledger and is managed in the form of a blockchain using a cryptographic hash function.

The medicine information management apparatus 200 may be referred to as a ‘Pharmaceutical Logistics System (PLS)’, and may be implemented in a form included in the logistics company 20 according to embodiments. When the private network 100 is implemented based on the Hyperledger Fabric blockchain network, the medicine information management apparatus 200 may be in charge of at least some of the functions of an orderer (ordering service node) in the Hyperledger Fabric blockchain network.

The medicine information management apparatus 200 includes a communication unit 210, a control unit 220, and a storage unit 230.

The communication unit 210 is configured to communicate with a plurality of supplier peers 111 and 112 mapped to a plurality of suppliers, a plurality of logistics company peers 120 mapped to a plurality of logistics companies 20, and a plurality of demander peers 130 mapped to a plurality of demander peers 30, which are approved to participate in the private network 100, through a wired or wireless communication network. For example, a wired communication network such as a local area network (LAN), a controller area network (CAN), or a daisy chain, and/or a wireless communication network such as Bluetooth, ZigBee, Wi-Fi, 5G, or the like may be utilized.

Each manufacturer 11 may manage at least one among a manufacturing/sales certificate, a Good Manufacturing Practice (GMP) evaluation report, and a serial number issued by a subject in charge (e.g., government) in a manufacturing region (e.g., country) of a medicine as a medicine identification value. Each manufacturer 11 may inquire medicine-related data recorded in the private network 100 by other peers (distributor peer 112, logistics company peer 120, demander peer 130) participating in the same channel through the manufacturer peer 111 using the manufacturer 11 identification data and authentication data. Each manufacturer 11 may request the manufacturer peer 111 mapped thereto to generate various commands (e.g., transactions) related to medicines through an application installed in the terminal of the manufacturer 11.

Each distributor 12 may share distribution information (e.g., delivery status of information) 41 medicines with each agreement subject through the private network 100 while classifying and managing other subjects (e.g., manufacturers 11, logistics companies 20) agreed to share information related to medicines with each other on the basis of their participant identification data. Each distributor 12 may request the distributor peer 112 mapped thereto to generate various commands related to medicine distribution information through an application installed in the terminal of the distributor 12.

Each logistics company 20 may record data on the transaction, management, and/or delivery of medicine units handled by logistics centers (storage warehouses) and delivery vehicles in the private network 100 through the logistics company peer 120, and allow peers of other subjects agreed to share information with the logistics company 20 to inquire the recorded data, on the basis of the medicine identification data mapped to the identification label of the medicine unit transferred from the manufacturer 11 and/or distributor 12. For reference, a plurality of peers mapped to a plurality of subjects agreed to share information may share the same medicine transaction book through the same channel in the private network 100. Each logistics company 20 may request the logistics company peer 120 mapped thereto to generate various commands related to medicines through an application installed in the terminal of the logistics company 20.

Each demander peer 130 may request the demander peer 130 mapped thereto to generate various commands related to medicines through an application installed in the terminal of the demander 30.

Each manufacturer peer 111 may inquire and/or update (record, delete) data in the medicine transaction book of a channel to which the manufacturer peer 111 belongs by executing a chain code mapped to a command received from the manufacturer 11 mapped thereto. Each distributor peer 112 may inquire and/or update (record, delete) data in the medicine transaction book of a channel to which the distributor peer 112 belongs by executing a chain code mapped to a command received from the distributor 12 (or the terminal of the distributor 12) mapped thereto. Each logistics company peer 120 may inquire and/or update (record, delete) data in the medicine transaction book of a channel to which the logistics company peer 120 belongs by executing a chain code mapped to a command received from the logistic company 20 (or the terminal of the logistic company 20) mapped thereto. Each demander peer 130 may inquire and/or update (record, delete) data in the medicine transaction book of a channel to which the demander peer 130 belongs by executing a chain code mapped to a command received from the demander 30 (or the terminal of the distributor 30) mapped thereto.

The control unit 220 may be implemented by hardware, firmware, combination thereof, and when it is implemented using hardware, it may be configured of application specific integrated circuits (ASIC) or digital signal processors (DSP), digital signal processing devices (DSPD), programmable logic devices (PLD), field programmable gate arrays (FPGA), and the like, and when it is implemented using firmware or software, the firmware or software may be configured to include modules, procedures, functions, or the like that perform the functions or operations described above.

The control unit 220 performs channel allocation and authority grant for a plurality of supplier peers 111 and 112, a plurality of logistics company peers 120, and a plurality of demander peers 130, using a result of authentication (participant authentication data) performed on a plurality of suppliers, a plurality of logistics companies 20, and a plurality of demanders 30 on the basis of the participant identification data provided by the plurality of supplier peers 111 and 112, the plurality of logistics company peers 120, and the plurality of demander peers 130. In this way, a channel to which each peer belongs and the authority of each peer are determined.

The storage unit 230 stores a computer program that allows the control unit 220 to execute a medicine information management method, and data and software related thereto. In addition, the storage unit 230 stores data generated as a result of an arithmetic operation of the control unit 220. The storage unit 230 may be implemented as read only memory (ROM), random access memory (RAM), erasable programmable read only memory (EPROM), electrically erasable programmable read-only memory (EEPROM), flash memory, static RAM (SRAM), a hard disk drive (HDD), solid state drive (SSD), or the like.

The storage unit 230 stores channel information that defines a channel to which each of a plurality of supplier peers 111 and 112, a plurality of logistics company peers 120, and a plurality of demander peers 130 belongs as a result of performing channel allocation. In addition, the storage unit 230 stores authority information that defines an inquiry right and an update right of each of a plurality of supplier peers 111 and 112, a plurality of logistics company peers 120, and a plurality of demander peers 130 as a result of performing authority grant.

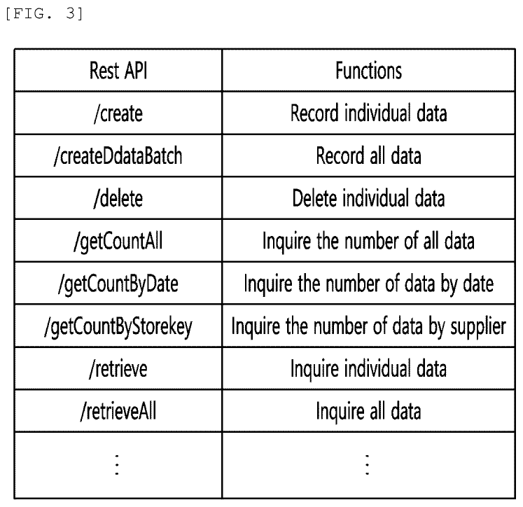

The control unit 220 may provide a plurality of Application Programming Interfaces (APIs) used for medicine information management to applications installed in the terminals of a plurality of suppliers, a plurality of logistics companies 20, and a plurality of demanders 30 through the plurality of supplier peers 111 and 112, the plurality of logistics company peers 120, and the plurality of demander peers 130, and FIG. 3 a view showing a list of a plurality of Rest (Representational State Transfer) APIs and their functions provided to the application of a participant (terminal of the participant).

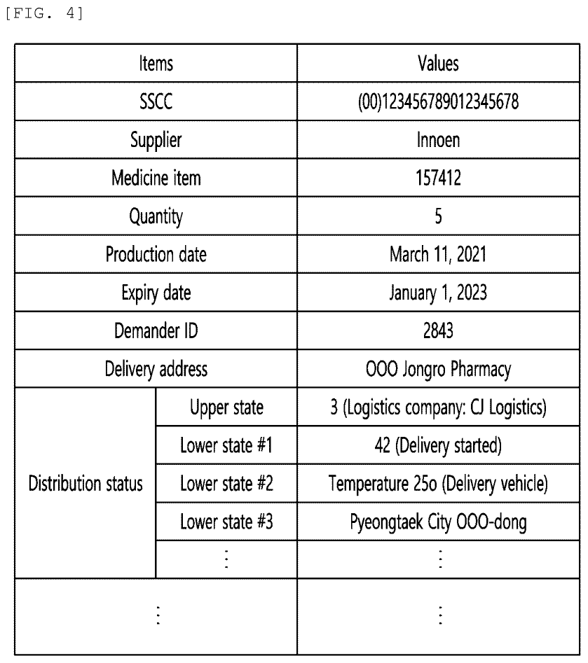

Each logistics company 20 may assign (issue) medicine identification data to each medicine unit corresponding to each medicine order of each supplier received by the logistics company 20. For example, the logistics company 20 may scan the barcode printed on the identification label (box label) attached to the medicine unit, and assign medicine identification data for tracking the medicine unit according to the scan data. FIG. 4 shows the structure of the medicine identification data. The medicine identification data includes a medicine identification value (e.g., Serial Shipping Container Code (SSCC)) assigned to the medicine unit and distribution status of the medicine unit. In addition to the SSCC, values according to various identification formats used in the distribution process, such as a batch number, barcode, QR code, EAN-13, GTIN-14, and the like, may be added as the medicine identification value. The distribution status may include an upper state and a lower state. The upper state has a value indicating a subject currently handling the medicine unit among the manufacturer 11, the distributor 12, the logistics company 20, and the demander 30. For example, whenever the medicine unit is transferred in order of the manufacturer 11, the distributor 12, the logistics company 20, and the demander 30, the value of the upper state is changed in order of 1, 2, 3, and 4. The lower state has a value indicating detailed distribution status of the medicine unit in a handling subject indicated by the upper state. For example, when the upper state indicates the logistics company 20, the lower state may indicate any one among inventory storage information (e.g., logistics center location, scheduled shipping date), delivery vehicle dispatch information (e.g., delivery person in charge, plate number of delivery vehicle), and delivery status information (e.g., events such as order reception, collection, loading on main line, unloading from main line, delivery started, current location, delivery completed, delivery cancelled, and the like, and occurrence time of each event). The medicine identification data may further include at least one among the name, quantity, production area, production date, export date, shipping date, expiry date, manufacturing certificate, Good Manufacturing Practice (GMP) evaluation report, and invoice of each medicine included in the medicine unit.

Meanwhile, the invoice includes delivery address information (e.g., name, geographical address, contact information of demander 30) for identifying the demander 30 of the medicine unit. At least two of the plurality of suppliers may assign different delivery address information to a specific demander 30. In this case, the specific demander 30 is unable to integrally confirm delivery status information (e.g., cold chain information) for each of the medicine units transferred to the logistics company 20 from two or more suppliers who have assigned different delivery address information to the specific demander 30 among several suppliers who supply medicines to the specific demander 30. The medicine information management apparatus 200 may unify delivery addresses registered to be different from each other for the specific demander 30 as a reference address by applying a delivery address unification algorithm to the delivery address information of the demanders 30 recorded in the medicine transaction book managed in the private network 100 by the channel. The reference address is an address previously input by the specific demander 30 as a delivery address mapped to its own demander identification data, or an address determined by the medicine information management apparatus 100 by comparing a plurality of delivery addresses recorded in the private network 200 for the specific demander 30. The demander 30 may be provided with distribution information for each medicine unit being delivered to the corresponding demander 30 extracted from the medicine transaction book including the reference address through the demander peer 130.

Each logistics company 20 may transmit the medicine identification data assigned to each medicine unit to the terminal of the logistics company 20 mapped thereto. The control unit 220 may generate a data stack including the medicine identification data in response to a request for recording the medicine identification data assigned to each medicine unit corresponding to a medicine order for each supplier received from each logistics company peer 120 by the logistics company 20 mapped to the corresponding logistics company peer 120, and then control the communication unit 210 to transmit the generated data stack to a channel to which the corresponding logistics company peer 120 belongs. Each data stack may include a header including a hash value (e.g., hash ID) of a preceding data stack and a hash value of the corresponding data stack, and a body including a plurality of transactions generated after the time point of generating the preceding data stack and sorted in order. When the private network 100 is implemented based on a Hyperledger Fabric blockchain network, the data stack may be referred to as a block.

Under this configuration, the block generated by the medicine information management apparatus 200 is transferred to all peers belonging to the corresponding channel in response to execution of the chain code performed by at least one of the peers belonging to each channel. The medicine information management apparatus 200 may generate a latest block to be linked to a genesis block or a preceding block on the basis of a consensus algorithm. The medicine information management apparatus 200 may allocate a channel to each peer on the basis of another consensus algorithm. The medicine information management apparatus 200 may grant an authority to each peer on the basis of still another consensus algorithm. Through this, when secrecy or confidentiality is required between organizations or peers within the same organization, the peers may be set to have a different inquiry right or update right.

In each channel, all the peers belonging to the corresponding channel may check validity (whether a predetermined warranty policy is observed) of all transactions in the data stack transmitted from the medicine information management apparatus 200. Each peer reflects (connects) a corresponding data stack to the medicine transaction book when all the transactions in the received data stack are valid. When at least one transaction in the received data stack is invalid, each peer may transmit an error message including an identification value of the invalid transaction to the medicine information management apparatus 200. For example, when the temperature information included in a specific transaction (e.g., storage temperature in a logistics center) does not satisfy the cold chain criterion, the transaction may be treated as invalid. The medicine information management apparatus 200 may confirm the medicine identification value (e.g., SSCC) included in the invalid transaction, and automatically reorder through the supplier peers 111 and 112 mapped to the supplier of the medicine unit having the confirmed medicine identification value, and additionally, transmit a delivery delay guide message through the demander peer 130 mapped to the demander 30 of the corresponding medicine unit.

Accordingly, as a plurality of peers participating in the same channel in the private network 100 and a plurality of subjects mapped to the peers mutually verify the integrity of the data recorded in the medicine transaction book managed through the same channel, and selectively incorporate only valid data into the medicine transaction book, information related to medicines may be managed transparently and safely throughout the entire distribution process from manufacturing to consumption.

The control unit 220 may extract medicine data filtered according to at least one criterion item from medicine transaction books managed through all the channels in the private network 100, and create a medicine information inquiry screen in which the extracted medicine data is displayed as at least one graphic object.

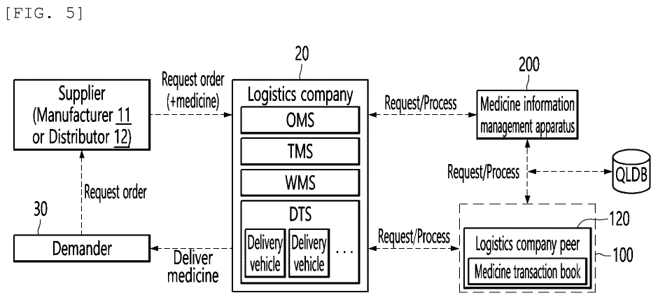

FIG. 5 is a view referenced to describe the operation of the medicine information management apparatus 200 for managing medicine information in association with a supplier, a logistics company 20, and a demander 30 belonging to the same channel in the private network 100. For convenience of explanation, one supplier, one logistics company 20, and one demander 30 are described.

Referring to FIG. 5, the demander 30 places an order of a medicine from a supplier. The supplier (manufacturer 11 or distributor 12) prepares a medicine unit attached with an identification label through bundle-packaging or the like for the ordered medicine, and provides the medicine unit to the logistics company 20, while transmitting a request for ordering the medicine placed by the demander 30 to the logistics company 20.

The logistics company 20 receives the medicine order from the supplier and collects a medicine unit corresponding thereto in a logistics center. At this point, medicine identification data is assigned to each medicine unit through scanning of a identification attached to the collected medicine unit.

The logistics company 20 may include an order management system (OMS), a transport management system (TMS), a warehouse management system (WMS), and a delivery tracking system (DTS).

The OMS collects the request for ordering the medicine transmitted from supplier, processes the collected order request, and determines whether or not to stock the medicine unit concentrated in the logistics center in response to the order request. The OSM may transmit a request for dispatching a delivery vehicle to the DTS for the medicine unit that has not been determined to stock.

The TMS transmits a request for ordering the medicine unit determined to stock by the OMS to the WMS. The TMS creates delivery vehicle dispatch information for allocating a transport vehicle for transporting the medicine unit determined to stock to the logistics warehouse linked to the WMS.

The WMS collects the request for ordering the medicine unit determined to stock from the OMS, processes the collected order request, and updates storage information on the medicine unit (e.g., storage location, storage quantity, storage temperature, storage humidity, storage period) stored in the warehouse. In addition, when an order for shipping the medicine unit stored in a warehouse is collected, the WMS may transmit, to the DTS, a request for dispatching a delivery vehicle for the medicine unit ordered to ship.

The DTS allocates a delivery vehicle for the medicine unit of each shipping order in response to the shipping order collected from the OMS and the WMS. The delivery vehicle periodically or non-periodically monitors delivery data (e.g., temperature, humidity, location) of the medicine unit until delivery of the medicine unit to the consumer 30 is completed. The delivery vehicle is equipped with various functions (e.g., temperature control, humidity control, location tracking) required to satisfy predetermined delivery conditions (e.g., cold chain) for the medicine unit assigned thereto. The DTS may collect delivery data of the medicine unit monitored by the delivery vehicle, and generate an update transaction for reflecting the collected delivery data to a channel to which the logistics company peer 120 mapped to the logistics company 20 belongs.

The logistics company peer 120 may receive a request for inquiring, updating, and/or deleting the delivery data generated for the medicine unit from the logistics company 20 (at least one among the OMS, TMS, WMS, and DTS), and receive a processing result (inquiry result, update result) for the requested content from the medicine information management apparatus 200 through communication with the medicine information management apparatus 200.

The medicine information management apparatus 200 may communicate with the logistics company peer 120, and return processing results of various requests related to the medicine unit transmitted from the supplier to the logistics company 20 to the logistics company peer 120. In addition, the medicine information management apparatus 200 may function as a mediator that allows a block, which includes delivery data frequently generated in the form of a transaction during delivery of the medicine unit performed by the logistics company 20, to be reflected in the medicine transaction books of all peers belonging to the same channel in the private network 100. When the private network 100 is implemented based on the Hyperledger Fabric blockchain network, a QLDB (Quantum Ledger Database) in which all medicine transaction books managed in all channels are integrally recorded may be provided inside one among the private network 100 and the medicine information management apparatus 200 or outside the private network 100 and the medicine information management apparatus 200. The QLDB guarantees integrity, completeness, and verifiability of the data of the medicine transaction books recorded therein, and permanently and completely stores data on the distribution process for each medicine unit associated with the participants of the private network 100.

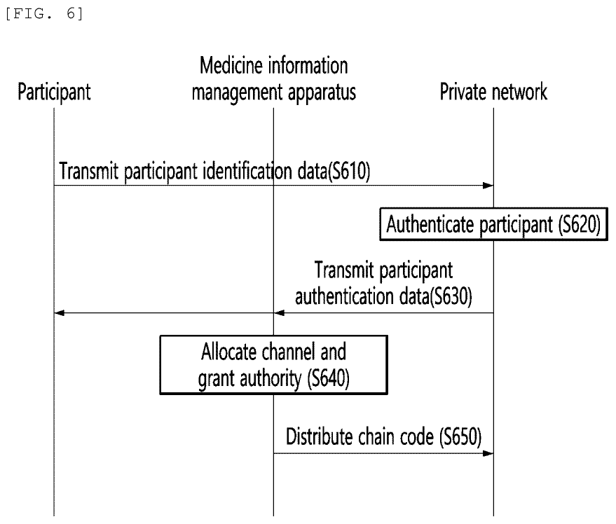

FIG. 6 is a flowchart referenced to describe the channel allocation operation and the authority grant operation performed by the medicine information management apparatus 200 for a peer mapped to each participant of the private network 100. In FIG. 6, the description of the participant may be commonly applied to all of the manufacturer 11, the distributor 12, the logistics company 20, and the demander 30.

Referring to FIG. 6, at step S610, the participant transmits participant identification data to the private network 100. The certificate authority peer (CA) of an organization to which the participant in the private network 100 belongs receives the participant identification data transmitted by the participant. The operation performed by the participant may be understood as an operation performed by the participant using a device possessed by the participant.

At step S620, the certificate authority peer (CA) of the private network 100 authenticates whether the participant is in a previously approved state to be able to participate in the private network 100 by applying predetermined authentication logic such as SNS authentication, DID authentication, or the like to the received participant identification data. In addition, the certificate authority peer (CA) of the private network 100 generates participant authentication data as a result of authenticating the participant. The participant authentication data may include, for example, the private key, public key, and/or affiliated organization code of the participant. The private key may function as an electronic signature of the participant used for encryption of data that can be processed in the private network 100. The public key may function as identification information of the participant or a peer mapped thereto. The affiliated organization code is used to identify an organization to which the participant belongs among a manufacturing organization, a distribution organization, a logistics organization, and a demand organization.

At step S630, the certificate authority peer (CA) of the private network 100 transmits the participant authentication data to the participant and the: medicine information management apparatus 200.

At step S640, the medicine information management apparatus 200 executes channel allocation and authority grant for a participant peer mapped to the participant on the basis of the participant authentication data. For example, when the participant is the manufacturer 11, the participant peer is the manufacturer peer 111. Specifically, the medicine information management apparatus 200 identifies at least one channel, among the channels constructed in the private network, that can be accessed by the participant peer, and allocates the identified channel to the participant peer, on the basis of a participant type (manufacturer 11, distributor 12, logistics company 20, or demander 30), channel permission rules among participants set in advance for all the participants, or the like. Similarly, the medicine information management apparatus 200 grants the participant peer an inquiry right that defines a category of data that the participant peer may inquire and an update right that defines a category of data that can be updated through a channel allocated to the participant peer, on the basis of a participant type, right permission rules among participants set in advance for all the participants, or the like.

At step S650, the medicine information management apparatus 200 stores, in the storage unit 230, channel information that defines a channel to which each of a plurality of supplier peers 111 and 112, a plurality of logistics company peers 120, and a plurality of demander peers 130 belongs as a result of performing channel allocation, and authority information that defines an inquiry right and an update right of each of a plurality of supplier peers 111 and 112, a plurality of logistics company peers 120, and a plurality of demander peers 130 as a result of performing authority grant.

At step S660, the medicine information management apparatus 200 distributes a chain code to the participant peer. The participant peer installs the chain code distributed from the medicine information management apparatus 200 in its storage. The chain code distributed to the participant peer corresponds to at least one transaction that can be requested by the participant to the participant peer according to the inquiry right and the update right determined at step S640. For example, when the participant has only a right to inquire the location of a medicine unit without a right to update, at step S650, the chain code distributed to the participant peer is executed to generate an inquiry transaction related to the distribution status such as the location of medicine.

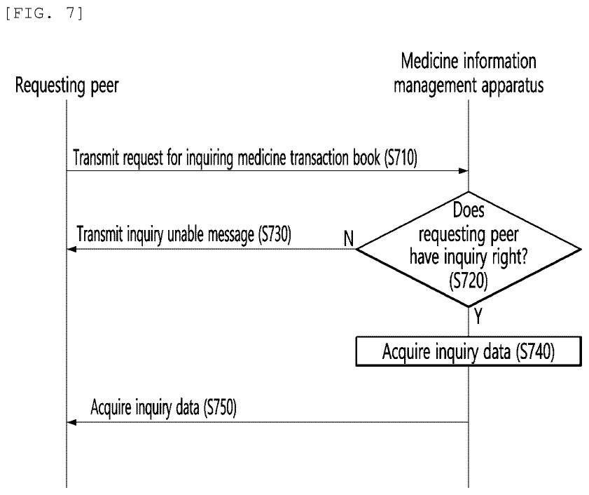

FIG. 7 is a flowchart referenced to describe a method of the medicine information management apparatus 200 for processing an inquiry request received from each peer of the private network 100. For convenience of explanation, a peer that generated an inquiry request is referred to as a requesting peer, and a subject (manufacturer 11, distributor 12, logistics company 20, or demander 30) mapped to the requesting peer is referred to as a requester.

Referring to FIG. 7, at step S710, the requesting peer executes a chain code for inquiry in response to an inquiry transaction generated by the requester, and transmits a request for inquiring the medicine transaction book to the medicine information management apparatus 200. For example, the requester is a demander 30, and the requesting peer is a demander peer 130. The inquiry request may include identification data of the demander peer 130, an order request made by the demander 30 to the supplier (or the identification value of the ordered medicine (e.g., SSCC)), and desired inquiry items (e.g., delivery status, current location, scheduled arrival time, and the like). The inquiry request is received by the communication unit 210 of the medicine information management apparatus 200.

At step S720, the control unit 220 of the medicine information management apparatus 200 verifies the inquiry right of the requesting peer. That is, the control unit 220 fetches the authority information recorded in the storage unit 230 and determines whether the requesting peer holds an inquiry right for each desired inquiry item included in the inquiry request. When the value at step S720 is “No (N)”, the process proceeds to step S730. When the value at step S720 is “Yes (Y)”, the process proceeds to step S740.

At step S730, the control unit 220 of the medicine information management apparatus 200 controls the communication unit 210 to transmit an inquiry unable message to the requesting peer. The inquiry unable message is a message informing that at least one desired inquiry item in the inquiry request has not been approved.

At step S740, the control unit 220 of the medicine information management apparatus 200 accesses a channel to which the requesting peer belongs through the communication unit 210, and acquires inquiry data for each desired inquiry item included in the inquiry request from the medicine transaction book of the channel.

At step S750, the control unit 220 of the medicine information management apparatus 200 controls the communication unit 210 to transmit the inquiry data obtained at step S740 to the requesting peer.

The requesting peer may directly acquire the inquiry data from the same medicine transaction book possessed by the requesting peer itself, in parallel to the process of acquiring inquiry data by the medicine information management apparatus 200 described above. In this case, the requesting peer may secure accuracy of the inquiry data by confirming whether the inquiry data acquired by the requesting peer itself matches the inquiry data received from the medicine information management apparatus 200 at step S750. Meanwhile, when the inquiry data does not match, this may be notified to the medicine information management apparatus 200. The medicine information management apparatus 200 may return to the step S720 described above in response to the mismatch event of the inquiry data.

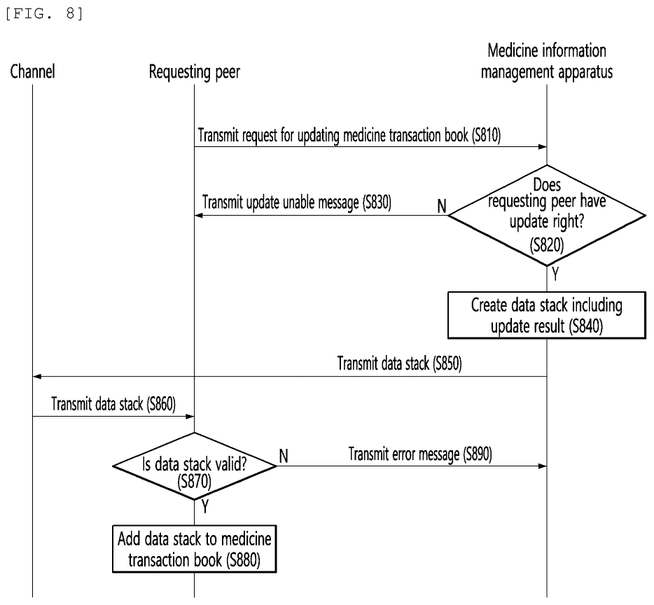

FIG. 8 is a flowchart referenced to describe a method of the medicine information management apparatus 200 for processing an update request received from each peer of the private network 100. In a way similar to FIG. 7, for convenience of explanation, a peer that generated an update request is referred to as a requesting peer, and a subject (manufacturer 11, distributor 12, logistics company 20, or demander 30) mapped to the requesting peer is referred to as a requester.

Referring to FIG. 8, at step S810, the requesting peer executes a chain code for update in response to an update transaction generated by the requester, and transmits a request for update the medicine transaction book to the medicine information management apparatus 200. For example, the requester is a logistic company 20, and the requesting peer is a logistic company peer 120. The update request may include identification data of the logistic company 20, desired update items (e.g., medicine identification data (SSCC), distribution status (temperature, humidity, location, delivery vehicle information), and an update type (e.g., log, delete). The update request is received by the communication unit 210 of the medicine information management apparatus 200.

At step S820, the control unit 220 of the medicine information management apparatus 200 verifies the update right of the requesting peer. That is, the control unit 220 fetches the authority information recorded in the storage unit 230 and determines whether the requesting peer holds an update right for each desired update item and the update type included in the update request. When the value at step S820 is “No (N)”, the process proceeds to step S830. When the value at step S820 is “Yes (Y)”, the process proceeds to step S840.

At step S830, the control unit 220 of the medicine information management apparatus 200 controls the communication unit 210 to transmit an update unable message to the requesting peer.

At step S840, the control unit 220 of the medicine information management apparatus 200 generates a data stack including an update result according to the update request. For example, when the update request of step S810 is a record of medicine identification data assigned to each medicine unit corresponding to the medicine order for each supplier received from the logistics company 20 mapped to the logistics company peer 120, a data stack including a transaction recording the medicine identification data may be created. As another example, when the update request of step S810 is a record of at least one item (e.g., temperature, location) indicating distribution status of the medicine unit, a data stack including a transaction recording a corresponding distribution status item may be created.

At step S850, the control unit 220 of the medicine information management apparatus 200 controls the communication unit 210 to transmit the data stack newly created at step S840 to a channel to which the requesting peer belongs.

At step S860, the channel transfers the data stack to the requesting peer. Of course, the data stack is transferred to all other peers belonging to the same channel, as well as the requesting peer.

At step S870, the requesting peer examines validity of the data stack. When the value at step S870 is “Yes (Y)”, the process proceeds to step S880. When the value at step S870 is “No (N)”, the process proceeds to step S890.

At step S880, the requesting peer updates the medicine transaction book by adding the data stack to the medicine transaction book.

At step S890, the requesting peer transmits an error message indicating that the data stack is invalid to the medicine information management apparatus 200. The error message may also be transferred to the requester through the requesting peer.

The control unit 220 may process the error message received at step S890 and confirm the content of the error message. When the content of the error message satisfies the requirements (e.g., temperature of the medicine goes out of the cold chain requirement during the distribution process) for reordering (cancellation of delivery) the medicine unit, the control unit 220 may transmit a distribution unsuitable message including an identification value of the corresponding medicine unit to a subject currently handling the medicine unit or a peer mapped thereto. In addition, the control unit 220 may automatically transmit a reorder request to at least one among the supplier and the demander 30 of the medicine unit.

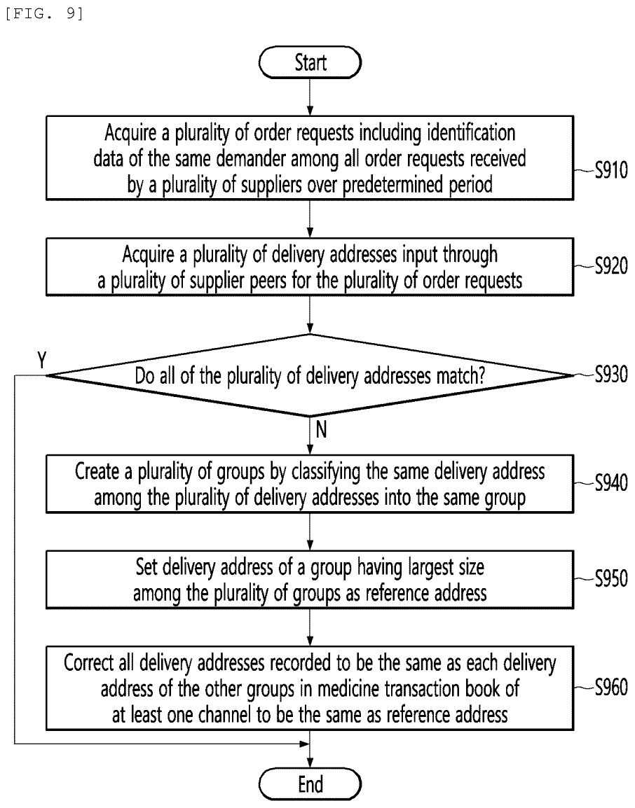

FIG. 9 is a flowchart referenced to describe a method of the medicine information management apparatus 200 for unifying delivery address information of the same demander 30 managed by a plurality of suppliers. The method of FIG. 9 may be periodically or non-periodically executed for each of the plurality of demanders 30 by the delivery address unification program installed in the medicine information management apparatus 200.

Referring to FIG. 9, at step S910, the control unit 220 acquires, from the medicine transaction book of at least one channel in the private network 100, a plurality of order requests including demander identification data of the same demander 30 among all order requests received by a plurality of suppliers over a predetermined period. For reference, the order request transmitted from the demander 30 to the supplier includes demander identification data of a corresponding demander 30.

At step S920, the control unit 220 acquires a plurality of delivery addresses input through a plurality of supplier peers 111 and 112 for the plurality of order requests acquired at step S910.

At step S930, the control unit 220 compares the plurality of delivery addresses acquired at step S920 and determines whether all of the plurality of delivery addresses match. When the value at step S930 is “No (N)”, the process proceeds to step S940.

At step S940, the control unit 220 creates a plurality of groups by classifying the same delivery address among the plurality of delivery addresses into the same group.

At step S950, the control unit 220 sets the delivery address of the group having the largest size among the plurality of groups as a reference address.

At step S960, the control unit 220 corrects all the delivery addresses recorded to be the same as each delivery address of the other groups in the medicine transaction book of at least one channel to be the same as the reference address.

Accordingly, there is an advantage in that although a plurality of suppliers may input different delivery addresses for the same demander 30, the demander 30 may integrally receive inquiry data on the delivery status (e.g., cold chain information) of each medicine ordered from a plurality of suppliers by using the identification data of its demander peer 130.

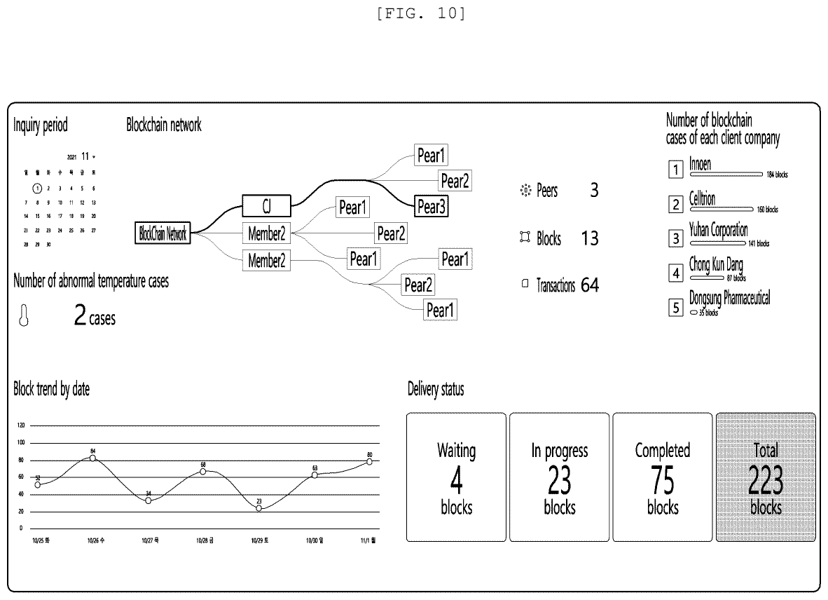

FIGS. 10 to 12 are views showing medicine information screens that can be provided in response to an inquiry request received from a participant of the private network 100.

Referring to FIG. 10, a participant may be provided with, within his or her inquiry right, a medicine information inquiry screen in which numerical information of blocks (data stacks) of each supplier of a supply organization is filtered by date, and the control unit 220 may generate medicine tracking information associated with each of a plurality of suppliers by date. The medicine tracking information may include at least one among the number of new orders, the number of update requests received for all medicine orders, the number of orders before delivery, the number of orders being delivered, and the number of orders that have been delivered. In FIG. 10, each block one-to-one corresponds to an order request for a medicine. Through the medicine information inquiry screen that displays medicine information by supplier and by date as shown in FIG. 10, the participant may confirm various information, such as the number of orders received by a specific supplier by date, the number of orders being delivered by date, the number of events for updating delivery status, and the number of orders that have been delivered.

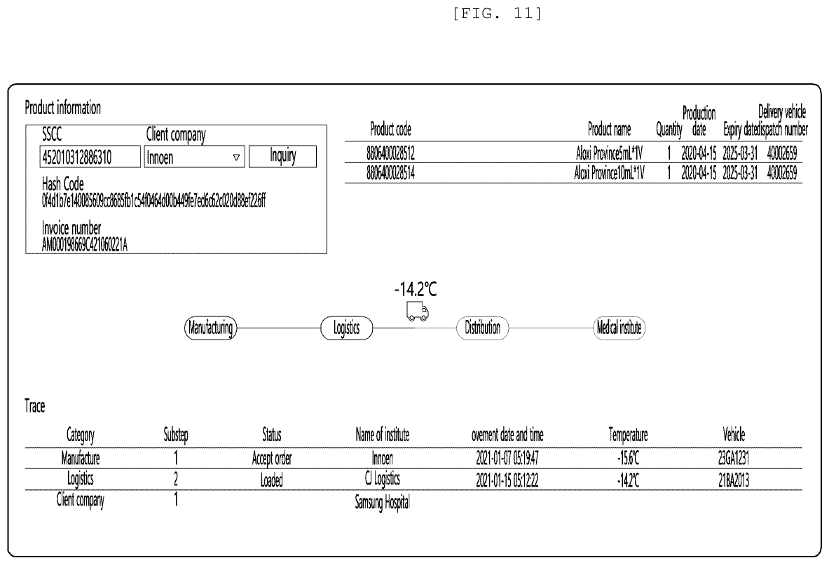

Referring to FIG. 11, a participant may be provided with, within his or her inquiry right, a medicine information inquiry screen showing delivery status of a medicine unit shipped by each supplier of a supply organization, and the control unit 220 may generate medicine tracking information for each medicine unit associated with each of a plurality of suppliers. The medicine tracking information may indicate an update event generated for the medicine unit and the time of generating the update event over a tracking period from the time of ordering the medicine unit to the present time. When the participant inputs the name of a supplier that the participant desires to inquire and the identification (e.g., SSCC) value of the medicine unit, a medicine information inquiry screen, on which graphic objects representing delivery history (e.g., delivery route, timeline (time) related to delivery route, temperature change, plate number of delivery vehicle) of each medicine unit are displayed, is provided as shown in FIG. 11.

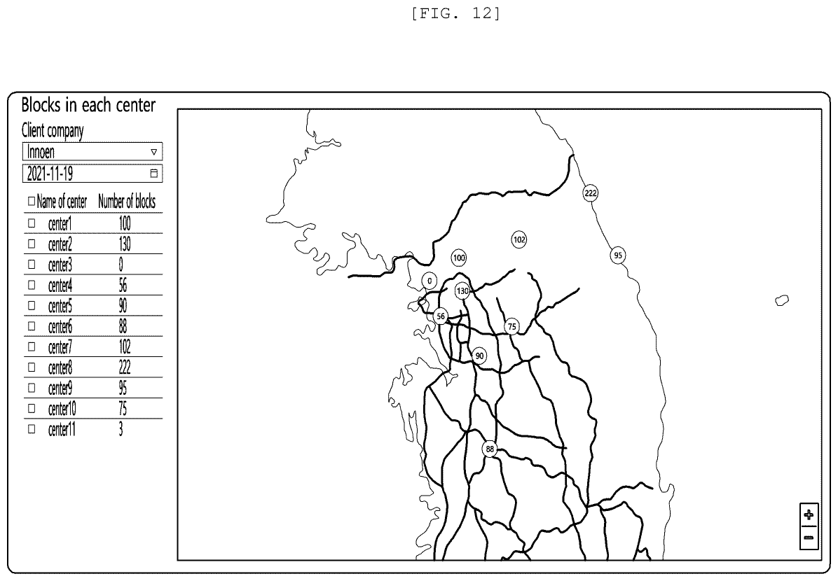

Referring to FIG. 12, a participant may be provided with, within his or her inquiry right, a medicine information inquiry screen showing by date the distribution status of all medicine units shipped by each supplier of a supply organization and distributed through a plurality of logistics companies 20 (logistics centers) in a predetermined region (e.g., Korea) in a distributed manner, and the control unit 220 may generate medicine tracking information of each date associated with each of a plurality of suppliers. In FIG. 12, the number of blocks associated with each logistics company 20 may be any one among the number of orders received on a specific date, the number of orders being delivered, and the number of orders that has been delivered, or a sum of two or more of them.

The present invention is not limited to the specific embodiments and applications described above, and various modifications can be made by those skilled in the art without departing from the gist of the present invention claimed in the claims, and these modified embodiments should not be understood separately from the technical spirit or perspective of the present invention.

Particularly, the configurations implementing the technical features of the present invention included in the block diagrams and flowcharts shown in the accompanying drawings mean logical boundaries between the configurations. However, according to an embodiment of software or hardware, since the illustrated configurations and their functions are implemented in the form of standalone software modules, monolithic software structures, codes, services, and combinations thereof, and the functions can be implemented by being stored in an executable medium in a computer having a processor capable of executing stored program codes, instructions, and the like, all of these embodiments should also be regarded as falling within the scope of the present invention.

Accordingly, although the accompanying drawings and description thereof describe the technical features of the present invention, they should not be simply inferred unless a specific arrangement of software for implementing these technical features is explicitly mentioned. That is, since various embodiments described above may exist, and such embodiments may be partially modified while retaining technical characteristics the same as those of the present invention, this should also be regarded as falling within the scope of the present invention.

In addition, in the case of flowcharts, although operations are described in the drawings in a specific order, they are illustrated to obtain the most desirable results, and it should not be understood that such operations should be performed in a shown specific order or in a sequential order, or all the illustrated operations should be performed. In a specific case, multitasking and parallel processing may be advantageous. In addition, separation of various system components in the embodiments described above should not be understood as being required in all the embodiments, and it should be understood that the described program components and systems may be generally integrated together into a single software product or packaged into multiple software products.

1. A medicine information management apparatus comprising:
a communication unit configured to communicate with a plurality of supplier peers mapped to a plurality of suppliers, a plurality of logistics company peers mapped to a plurality of logistics companies, and a plurality of demander peers mapped to a plurality of demander peers, which are approved to participate in a private network;
a control unit for performing channel allocation and authority grant for the plurality of supplier peers, the plurality of logistics company peers, and the plurality of demander peers, using a result of authentication performed on the plurality of suppliers, the plurality of logistics companies, and the plurality of demanders on the basis of participant identification data provided by the plurality of supplier peers, the plurality of logistics company peers, and the plurality of demander peers; and
a storage unit for storing channel information that defines a channel to which each of the plurality of supplier peers, the plurality of logistics company peers, and the plurality of demander peers belongs as a result of performing the channel allocation, and authority information that defines an inquiry right and an update right of each of the plurality of supplier peers, the plurality of logistics company peers, and the plurality of demander peers as a result of performing the authority grant.

2. The apparatus according to claim 1, wherein the control unit generates a data stack including medicine identification data in response to an update request for registering the medicine identification data assigned to each medicine unit corresponding to a medicine order for each supplier received from each logistics company peer by a logistics company mapped to a corresponding logistics company peer, and then controls the communication unit to transmit the data stack to a channel to which the corresponding logistics company peer belongs so that the data stack is added to medicine transaction books of all participants of the channel to which the corresponding logistics company peer belongs.

3. The apparatus according to claim 2, wherein the medicine identification data includes: a serial shipping container code (SSCC) assigned to the medicine unit; and distribution status of the medicine unit, and further includes at least one among a name, quantity, production area, production date, export date, shipping date, expiry date, manufacturing certificate, Good Manufacturing Practice (GMP) evaluation report, and invoice of each medicine included in the medicine unit.

4. The apparatus according to claim 2, wherein when a query request for the medicine transaction book is received from a requesting peer that is any one among the plurality of supplier peers, the plurality of logistics company peers, and the plurality of demander peers, the control unit verifies an inquiry right of the requesting peer for the medicine transaction book using the authority information and the channel information, and transmits inquiry data acquired from the medicine transaction book to the requesting peer in response to the inquiry request when the inquiry right of the requesting peer is verified.

5. The apparatus according to claim 4, wherein the control unit generates medicine tracking information associated with each of the plurality of suppliers by date, wherein the medicine tracking information includes at least one among the number of new orders, the number of update requests received for all medicine orders, the number of orders before delivery, the number of orders being delivered, and the number of orders that have been delivered.

6. The apparatus according to claim 4, wherein the control unit generates medicine tracking information for each medicine unit associated with each of the plurality of suppliers, wherein the medicine tracking information indicates an update event generated for the medicine unit and a time of generating the update event over a tracking period from a time of ordering the medicine unit to a present time.

7. The apparatus according to claim 2, wherein the control unit verifies, in response to a request for updating distribution status of the medicine unit received from a requesting peer that is any one among the plurality of supplier peers, the plurality of logistics company peers, and the plurality of demander peers, an update right of the requesting peer using the authority information and the channel information, updates the distribution status of the medicine unit according to the update request when the update right of the requesting peer is verified, and controls the communication unit to transmit a new data stack including an update result of the distribution status to a channel to which the requesting peer belongs so that the new data stack is added to the medicine transaction book.

8. The apparatus according to claim 7, wherein the distribution status indicates at least one among inventory storage information, delivery vehicle dispatch information, delivery status information, and temperature information of the medicine unit.

9. A medicine information management method comprising the steps of:
performing channel allocation and authority grant for a plurality of supplier peers, a plurality of logistics company peers, and a plurality of demander peers, using a result of authentication performed on a plurality of suppliers, a plurality of logistics companies, and a plurality of demanders on the basis of participant identification data provided by
the plurality of supplier peers mapped to the plurality of suppliers, the plurality of logistics company peers mapped to the plurality of logistics companies, and the plurality of demander peers mapped to the plurality of demander peers, which are approved to participate in a private network; and
storing channel information that defines a channel to which each of the plurality of supplier peers, the plurality of logistics company peers, and the plurality of demander peers belongs as a result of performing the channel allocation, and authority information that defines an inquiry right and an update right of each of the plurality of supplier peers, the plurality of logistics company peers, and the plurality of demander peers as a result of performing the authority grant.

10. The method according to claim 9, further comprising the steps of:
generating a data stack including medicine identification data in response to an update request for registering the medicine identification data assigned to each medicine unit corresponding to a medicine order for each supplier received from each logistics company peer by a logistics company mapped to a corresponding logistics company peer; and
transmitting the data stack to a channel to which the corresponding logistics company peer belongs so that the data stack is added to medicine transaction books of all participants of the channel to which the corresponding logistics company peer belongs.

11. The method according to claim 10, wherein the medicine identification data includes: a serial shipping container code (SSCC) assigned to the medicine unit; and distribution status of the medicine unit, and further includes at least one among a name, quantity, production area, production date, export date, shipping date, expiry date, manufacturing certificate, Good Manufacturing Practice (GMP) evaluation report, and invoice of each medicine included in the medicine unit.

12. The method according to claim 10, further comprising the steps of:
receiving a query request for the medicine transaction book from a requesting peer that is any one among the plurality of supplier peers, the plurality of logistics company peers, and the plurality of demander peers;
verifying an inquiry right of the requesting peer for the medicine transaction book using the authority information and the channel information; and
transmitting inquiry data acquired from the medicine transaction book to the requesting peer in response to the inquiry request when the inquiry right of the requesting peer is verified.

13. The method according to claim 12, further comprising the step of generating medicine tracking information associated with each of the plurality of suppliers by date, wherein the medicine tracking information includes at least one among the number of new orders, the number of update requests received for all medicine orders, the number of orders before delivery, the number of orders being delivered, and the number of orders that have been delivered.

14. The method according to claim 12, further comprising the step of generating medicine tracking information for each medicine unit associated with each of the plurality of suppliers, wherein the medicine tracking information indicates an update event generated for the medicine unit and a time of generating the update event over a tracking period from a time of ordering the medicine unit to a present time.

15. The method according to claim 10, further comprising the steps of:
verifying, in response to a request for updating distribution status of the medicine unit received from a requesting peer that is any one among the plurality of supplier peers, the plurality of logistics company peers, and the plurality of demander peers, an update right of the requesting peer using the authority information and the channel information;
updating the distribution status of the medicine unit according to the update request when the update right of the requesting peer is verified; and
transmitting a new data stack including an update result of the distribution status to a channel to which the requesting peer belongs so that the new data stack is added to the medicine transaction book.
